(19) United States
(12) Patent Application Publication
(10) Pub. No.: US 2005/0287607 A1
(43) Pub. Date: Dec. 29, 2005

(54) Methods and compositions for identification of antigen-specific B cell populations

(76) Inventors: Betty A. Diamond, Bronx, NY (US); Jeffrey Newman, Sacramento, CA (US)

Correspondence Address: AMSTER, ROTHSTEIN & EBENSTEIN LLP, 90 PARK AVENUE, NEW YORK, NY, 10016, US

(21) Appl. No.: 11/029,213

(22) Filed: Jan. 4, 2005

Related U.S. Application Data
(60) Provisional application No. 60/534,655, filed on Jan. 7, 2004.

(51) Int. Cl. G01N 33/53

(52) U.S. Cl. 435/7.5

(57) ABSTRACT

Provided are compositions and methods for identifying, enumerating and isolating B cells that react to a particular antigen. Included are tetrameric antigen-avidin complexes not comprising an MHC molecule, kits for producing the complexes, and reagents for diagnosing an autoimmune disease or monitoring progression of an autoimmune disease comprising the complexes. Also provided are B cells comprising the complexes and methods of using the complexes for: labeling a B cell reactive to a specific antigen, isolating a B cell reactive to a specific antigen from a mixture of B cells reactive to more than one antigen, quantifying B cells reactive to a specific antigen in a mixture of B cells reactive to more than one antigen, measuring immunity in a mammal characterized by the presence of B cells reactive to a specific antigen, and monitoring a disease in a mammal or determining whether a mammal has a disease characterized by the presence of B cells reactive to a specific antigen.

### CROSS-REFERENCE TO RELATED APPLICATION

This application claims the benefit of U.S. Provisional Application No. 60/534,655, filed Jan. 7, 2004.

### BACKGROUND OF THE INVENTION

(1) Field of the Invention

The present invention generally relates to identification, enumeration and isolation of B cell populations that react to a particular antigen. More specifically, the invention relates to tetrameric antigen compositions that label B cells reacting to the antigen, allowing identification, enumeration and/or isolation of that B cell population.

(2) Description of the Related Art

### References Cited

Ando, D. G., F. M. Ebling and B. H. Hahn. Detection of native and denatured DNA antibody forming cells by the enzyme-linked immunospot assay. A clinical study of (New Zealand black×New Zealand white) 1 mice. Arthritis Rheum. 29 (1986), pp. 1139-1146.

Bynoe, M. S., L. Spatz and B. Diamond. Characterization of anti-DNA B cells that escape negative selection. Eur. J. Immunol. 29 (1999), pp. 1304-1313.

Bynoe, M. S., C. M. Grimaldi and B. Diamond. Estrogen up-regulates Bcl-2 and blocks tolerance induction of naive B cells. Proc. Natl. Acad. Sci. U.S.A. 97 (2000), pp. 2703-2708.

Chen, C., M. Z. Radic, J. Erikson, S. A. Camper, S. Litwin, R. R. Hardy and M. Weigert. Deletion and editing of B cells that express antibodies to DNA. J. Immunol. 152 (1994), pp. 1970-1982.

Chen, C., E. L. Prak and M. Weigert. Editing disease-associated autoantibodies. Immunity 6 (1997), pp. 97-105.

Cosenza, H., J. Quintans and I. Lefkovits. Antibody response to phosphorylcholine in vitro: I. Studies on the frequency of precursor cells, average clone size and cellular cooperation. Eur. J. Immunol. 5 (1975), pp. 343-349.

Erikson, J., M. Z. Radic, S. A. Camper, R. R. Hardy, C. Carmack and M. Weigert. Expression of anti-DNA immunoglobulin transgenes in non-autoimmune mice. Nature 349 (1991), pp. 331-334.

Friedmann, D., N. Yachimovich, G. Mostoslavsky, Y. Pewzner-Jung, A. Ben Yehuda, K. Rajewsky and D. Eilat. Production of high affinity autoantibodies in autoimmune New Zealand Black/New Zealand white F1 mice targeted with an anti-DNA heavy chain. J. Immunol. 162 (1999), pp. 4406-4416.

Gay, D., T. Saunders, S. Camper and M. Weigert. Receptor editing: an approach by autoreactive B cells to escape tolerance. J. Exp. Med. 177 (1993), pp. 999-1008.

Gaynor, B., C. Putterman, P. Valadon, L. Spatz, M. D. Scharff and B. Diamond. Peptide inhibition of glomerular deposition of an anti-DNA antibody. Proc. Natl. Acad. Sci. U.S.A. 94 (1997), pp. 1955-1960.

Gilkeson, G. S., K. Bernstein, A. M. Pippen, S. H. Clarke, T. Marion, D. S. Pisetsky, P. Ruiz and J. B. Lefkowith. The influence of variable-region somatic mutations on the specificity and pathogenicity of murine monoclonal anti-DNA antibodies. Clin. Immunol. Immunopathol. 76 (1995), pp. 59-67.

Goodnow, C. C., J. Crosbie, S. Adelstein, T. B. Lavoie, S. J. Smith-Gill, R. A. Brink, H. Pritchard-Briscoe, J. S. Wotherspoon, R. H. Loblay and K. Raphael. Altered immunoglobulin expression and functional silencing of self-reactive B lymphocytes in transgenic mice. Nature 334 (1988), pp. 676-682.

Harris, S. L., M. K. Park, M. H. Nahm and B. Diamond. Peptide mimic of phosphorylcholine, a dominant epitope found on Streptococcus pneumoniae. Infect. Immun. 68 (2000), pp. 5778-5784.

Harris, S., A. Dagtas and B. Diamond. Regulating the isotypic and idiotypic profile of an anti-PC antibody response: lessons from peptide mimics. Mol. Immunol. 39 (2002), pp. 263-272.

Ibrahim, S. M., M. Weigert, C. Basu, J. Erikson and M. Z. Radic. Light chain contribution to specificity in anti-DNA antibodies. J. Immunol. 155 (1995), pp. 3223-3233.

Iliev, A., L. Spatz, S. Ray and B. Diamond. Lack of allelic exclusion permits autoreactive B cells to escape deletion. J. Immunol. 153 (1994), pp. 3551-3556.

Kang, C. Y., T. K. Brunck, T. Kieber-Emmons, J. E. Blalock and H. Kohler. Inhibition of self-binding antibodies (autobodies) by a VH-derived peptide. Science 240 (1988), pp. 1034-1036.

Khalil, M. l, K. Inaba, R. Steinman, J. Ravetch and B. Diamond. T cell studies in a peptide-induced model of systemic lupus erythematosus. J. Immunol. 166 (2001), pp. 1667-1674.

Klinman, D. M., A. Shirai, Y. Ishigatsubo, J. Conover and A. D. Steinberg. Quantitation of IgM- and IgG-secreting B cells in the peripheral blood of patients with systemic lupus erythematosus. Arthritis Rheum. 34 (1991), pp. 1404-1410.

Kohler, G. and C. Milstein. Continuous cultures of fused cells secreting antibody of predefined specificity. Nature 256 (1975), pp. 495-497.

Lin, H. and R. Grosschedl. Failure of B-cell differentiation in mice lacking the transcription factor EBF. Nature 376 (1995), pp. 263-267.

Madaio, M. P., J. Carlson, J. Cataldo, A. Ucci, P. Migliorini and O. Pankewycz. Murine monoclonal anti-DNA antibodies bind directly to glomerular antigens and form immune deposits. J. Immunol. 138 (1987), pp. 2883-2889.

McHeyzer-Williams, L. J., M. Cool and M. G. McHeyzer-Williams. Antigen-specific B cell memory: expression and replenishment of a novel b220(-)memory b cell compartment. J. Exp. Med. 191 (2000), pp. 1149-1166.

Peeva, E., C. Grimaldi, L. Spatz and B. Diamond. Bromocriptine restores tolerance in estrogen-treated mice. J. Clin. Invest. 106 (2000), pp. 1373-1379.

Putterman, C. and B. Diamond. Immunization with a peptide surrogate for double-stranded DNA (dsDNA) induces autoantibody production and renal immunoglobulin deposition. J. Exp. Med. 188 (1998), pp. 29-38.

Ray, S. K., C. Putterman and B. Diamond. Pathogenic autoantibodies are routinely generated during the response to foreign antigen: a paradigm for autoimmune disease. Proc. Natl. Acad. Sci. U.S.A. 93 (1996), pp. 2019-2024.

Schmidt, K. N., C. W. Hsu, C. T. Griffin, C. C. Goodnow and J. G. Cyster. Spontaneous follicular exclusion of SHP1-deficient B cells is conditional on the presence of competitor wild-type B cells. J. Exp. Med. 187 (1998), pp. 929-937.

Spatz, L, A. Iliev, V. Saenko, L. Jones, M. Irigoyen, A. Manheimer-Lory, B. Gaynor, C. Putterman, M. Bynoe, C. Kowal, P. Kuo, J. Newman and B. Diamond. Studies on the structure, regulation, and pathogenic potential of anti-dsDNA antibodies. Methods 11 (1997), pp. 70-78.

The low frequency of antigen-specific B cells participating in protective antibody responses or in antibody-mediated autoimmune disorders presents a major difficulty in their functional and molecular characterization. In SLE, for instance, the frequency of B cells reactive to double-stranded (ds) DNA has been estimated to be 1 in 10,000 B cells in the peripheral blood of lupus patients (Klinman et al., 1991) and 1 in 1000 B cells in spleens of lupus-prone mice (Ando et al., 1986). Similarly, the anti-phosphorylcholine (PC) response which can protect mice from a lethal pneumococcal infection is mediated by fewer than 1 in 10,000 B cells (Cosenza et al., 1975). Even the murine response to sheep red blood cells, which is comprised of multiple antigenic specificities, engages no more than 1 in 100 splenic B cells (Kohler and Milstein, 1975).

To overcome the obstacles imposed by the low frequency of antigen-specific B lymphocytes, investigators have historically employed two approaches, either alone or in combination: hybridoma technology to capture autoreactive B cells, or genetically manipulated animals with an enhanced number of antigen-specific B cells (Madaio et al., 1987; Goodnow et al., 1988; Erikson et al., 1991; Gay et al., 1993; Chen et al., 1994, 1997; Iliev et al., 1994; Gilkeson et al., 1995; Ibrahim et al., 1995; Bynoe et al., 1999). While experiments using these techniques form the basis of our current understanding of B cell repertoire and activation, these approaches have limitations. Hybridomas are not necessarily representative of the entire B cell population and cannot be generated from anergic or pre-apoptotic cells using conventional methodology (Ray et al., 1996). Genetically altered mice may have abnormalities in B cell development. Furthermore, it has been demonstrated that cell fate is determined through competition with other cells. In the presence of a native repertoire of B cells, autoreactive anergic B cells do not enter lymphoid follicles; in their absence, the migration pattern is altered (Schmidt et al., 1998). In addition, the expression of a transgene may cause alterations in the B cell subsets of an antigen-specific response. Thus, the skewing of B cell specificities that occurs in mice expressing a transgene-encoded antibody may change B cell development and function.

Thus, there is a need for methods for determining the presence or quantity of B cells reactive to a particular antigen. The present invention satisfies that need.

### SUMMARY OF THE INVENTION

Accordingly, the inventors have succeeded in developing a method of identifying, enumerating and isolating B cells that react to a particular antigen. The method uses novel tetrameric antigen-avidin complexes.

Thus, in some embodiments, the invention is directed to tetrameric antigen-avidin complexes not comprising an MHC molecule.

The invention is also directed to mammalian B cells comprising a tetrameric antigen-avidin complex not comprising an MHC molecule.

In additional embodiments, the invention is directed to kits comprising an avidin and instructions for synthesizing a tetrameric antigen-avidin complex not comprising an MHC molecule.

The present invention is additionally directed to reagents for diagnosing an autoimmune disease or monitoring progression of an autoimmune disease. The reagents comprise a tetrameric antigen-avidin complex not comprising an MHC molecule, where the antigen is a dsDNA mimetope.

The invention is also directed to mixtures of B cells comprising B cells reactive to a specific antigen and B cells not reactive to a specific antigen. In these embodiments, the B cells reactive to the specific antigen are labeled with a tetrameric antigen-avidin complex not comprising an MHC molecule.

The present invention is further directed to methods of labeling a B cell reactive to a specific antigen. The methods comprise combining the B cell with a tetrameric antigen-avidin complex not comprising an MHC molecule. In these embodiments, the tetrameric antigen-avidin complex comprises the specific antigen and a detectable moiety.

Additionally, the invention is directed to methods of isolating a B cell reactive to a specific antigen from a mixture of B cells reactive to more than one antigen. The methods comprise combining the mixture of B cells with a tetrameric antigen-avidin complex not comprising an MHC molecule, where the tetrameric antigen-avidin complex comprises the specific antigen and a fluorescent moiety, then isolating the B cell with a fluorescence-activated cell sorter.

In additional embodiments, the invention is directed to methods of quantifying B cells reactive to a specific antigen in a mixture of B cells reactive to more than one antigen. The methods comprise combining the mixture of B cells with a tetrameric antigen-avidin complex not comprising an MHC molecule, where the tetrameric antigen-avidin complex comprises the specific antigen and a detectable moiety, then counting the B cells comprising the detectable moiety.

The present invention is also directed to methods of measuring immunity in a mammal characterized by the presence of B cells reactive to a specific antigen. The methods comprise combining B cells from the mammal with a tetrameric antigen-avidin complex not comprising an MHC molecule, where the tetrameric antigen-avidin complex comprises the specific antigen and a detectable moiety, then identifying B cells bound to the complex. In these methods, the B cells bound to the complex are B cells reactive to the specific antigen.

In further embodiments, the invention is directed to methods of monitoring a disease in a mammal or determining whether a mammal has a disease, where the disease is characterized by the presence of B cells reactive to a specific antigen. The methods comprise combining B cells from the mammal with a tetrameric antigen-avidin complex not comprising an MHC molecule, where the tetrameric antigen-avidin complex comprises the specific antigen and a detectable moiety, then identifying B cells bound to the complex. In these methods, the presence of B cells bound to the complex indicates the presence of the disease in the mammal.

### BRIEF DESCRIPTION OF THE DRAWINGS

FIG. 1 shows the results of experiments involving surface staining of splenocytes with R4A-tetramer and anti-dsDNA ELISpot assays on LPS-stimulated, tetramer-peptide-enriched splenocytes. Mice were immunized three times as described in the Example. Contours are plotted on a log scale (ns=not significant; Mo=monocyte; Ly=lymphocyte; PEP=peptide). Panel A shows the gating strategy for identification of tetramer-reactive lymphocytes. Panel B shows representative staining patterns of MAP-peptide and MAP-core-immunized mice with and without inhibitors. Percentages given are based on the number of events on each plot. Panel C shows the enumeration of anti-dsDNA AFCs following R4A-tetramer enrichment.

FIG. 2 shows the results of experiments involving surface staining of splenocytes with 10-2-tetramer and peptide 1-tetramer. Mice were immunized three times with 10-2-BSA or peptide 1-BSA, as described in the Example. Percentages given are based on the number of events on each plot, and shown on a log scale. Gating strategy is consistent with FIG. 1A. Panel A shows representative 10-2- and peptide 1-tetramer staining patterns of 10-2-BSA and peptide 1-BSA-immunized mice with and without inhibitor. Panel B shows representative PC-BSA staining patterns of 10-2-BSA and peptide 1-BSA-immunized mice with and without inhibitor.

FIG. 3 is graphs of experimental results of experiments involving enrichment of anti-peptide 1 activity by peptide 1-tetramers. In the experiment of Panel A, splenocytes were sorted for peptide 1 reactivity, and assayed for anti-peptide 1-secreting B cells. Unsorted and peptide 1-depleted had significantly lower tetramer-reactive B cells (p<0.0001). In the experiment of Panel B, splenocytes were sorted for peptide 1 reactivity and cultured overnight. Supernatants were assayed for total IgG and anti-peptide antibody (p<0.000l ).

FIG. 4 is a photograph and graph of CD79b expression in splenocytes sorted on peptide-specificity and B220. Panel A is a photograph of the results of multiplex PCR for 18S rRNA internal standard (488 bp, upper band) and CD79b (350 bp, lower band). Lanes 1-4 (left to right): peptide⁺ B220^{low}, peptide⁺ B220^{high}, peptide⁻ B220^{low}, peptide⁻ B220^{high}. Panel B is a graph showing relative RNA expression levels in splenocytes, shown as a ratio of CD79b versus 18S rRNA internal standard.

### DETAILED DESCRIPTION OF THE INVENTION

The present invention is directed to novel compositions and methods useful for identifying, enumerating and isolating B cells that react to a particular antigen. The novel compositions comprise tetrameric antigen complexes not comprising an MHC molecule. These complexes efficiently bind to B cells that specifically react to the antigen. See the Example.

Without being bound to any particular mechanism, it is believed that the tetrameric form of the complexes provide enhanced avidity for antigen binding, allowing the efficient and specific binding of the complex to B cells reactive to the specific antigen.

Thus, in some embodiments, the invention is directed to tetrameric antigen complexes not comprising an MHC molecule. These complexes can be made by any method known in the art, including using synthetic or natural linker molecules to bind the four antigens into a tetrameric complex. In preferred embodiments, the complexes are created by biotinylating the antigen, then combining the biotinylated antigen with an avidin. Thus, the tetrameric antigen complexes are preferably tetrameric antigen-avidin complexes.

As used herein, “avidin” is a protein with 4 high affinity (i.e., k_{d}<10⁻^{¹⁰} M) binding sites for d-biotin (“biotin”). The avidin can be naturally occurring, such as egg white avidin or streptavidin, or it may be one of the many known modified avidins, such as deglycosylated forms, including neutravidin, and nitrated forms, including captavidin, which have reduced biotin affinity at high pH. See, e.g., the avidins sold by Molecular Probes, Eugene Oreg.

The present invention is not narrowly limited to the use of any particular avidin. The skilled artisan with knowledge of the avidin literature could select an avidin for any particular application without undue experimentation. In many preferred embodiments, the avidin is streptavidin or neutravidin.

In preferred embodiments, the complex is labeled with a bindable or preferably, a detectable moiety, in order to more easily identify B cells to which the complex is bound. The invention is not limited to any particular bindable or detectable moiety, which can be selected by the skilled artisan without undue experimentation for any particular application. Examples of bindable moieties include a His6 tag or a second antigen or hapten that can bind an available antibody. In some embodiments, these moieties can aid in the isolation of B cells that have bound to the complex.

Examples of detectable moieties include radioactive moieties including, but not limited to, ³H, ¹⁴C, ³²P, and ¹²⁵I; a second antigen or hapten; or a fluorescent protein or molecule. For most embodiments, preferred detectable moieties are fluorescent dyes such as fluoresceine isothiocyanate (“FITC”), allophycocyanin, R-phycoerythrin, any of the various rhodamine derivatives, etc.

The antigen in the complex can be any antigen, including any peptide, protein, carbohydrate, or hapten antigen, that can be biotinylated. Nonlimiting examples of useful antigens include antigens targeted in an allergic reaction, vaccine antigens, microbial antigens and viral antigens. The complex is particularly useful when employing a mammalian antigen targeted in an autoimmune disease, such as lupus. A particularly useful autoimmune antigen is a peptide dsDNA mimetope, such as the R4A peptide provided herein as SEQ ID NO:1. A complex employing the R4A peptide, particularly when labeled with a fluorescent molecule, is especially useful for various methods related to lupus diagnosis and therapy, as described below.

The antigen-avidin complex of the present invention is preferably produced by biotinylating the antigen, by known methods, then combining the biotinylated antigen with the avidin. If a labeled complex (i.e., with a detectable moiety such as a fluorescent dye) is desired, the label can be conjugated to the complex either after, or preferably before, combining the biotinylated antigen with the avidin. In this regard, various avidins labeled with any of numerous fluorescent dyes are available from several commercial sources (e.g., Molecular Probes); additional avidin-label combinations can be produced without undue experimentation.

The present invention is also directed to mammalian B cells comprising any of the above-described tetrameric antigen-avidin complexes. In preferred embodiments, the B cell is a human B cell, since the identification of such cells are useful for evaluation of human disease. In order to more easily identify the B cells comprising the complex, it is preferred that the complex further comprises a detectable moiety, preferably a fluorescent moiety.

Where the mammalian cell is from a mammal with an autoimmune disease, e.g., lupus, the antigen in the complex is preferably a dsDNA mimetope, such as the peptide provided herein as SEQ ID NO:1.

In some embodiments, e.g., when B cells (for example in a peripheral blood mononuclear cell [PBMC] composition) from a mammal are tested for reactivity to the antigen in the complex, the B cell comprising the complex can be in a composition further comprising B cells that do not comprise the complex. The B cell comprising the complex can also be isolated from B cells not comprising the complex, for example using a fluorescence-activated cell sorter (FACS).

In other embodiments, the invention is directed to kits comprising an avidin and instructions for synthesizing a tetrameric antigen-avidin complex not comprising an MHC molecule. The avidin can optionally further comprise a detectable moiety, preferably a fluorescent dye. The kits can also further comprise other reagents useful for synthesizing the complex, such as a reagent for biotinylation of an antigen.

The present invention is also directed to reagents for diagnosing an autoimmune disease or monitoring progression of an autoimmune disease. The reagents comprise the above-described tetrameric antigen-avidin complex, where the antigen is a dsDNA mimetope. The complex preferably further comprises a detectable moiety, most preferably a fluorescent dye. In other preferred embodiments, the the autoimmune disease is lupus and the antigen comprises SEQ ID NO:1 (i.e., the R4A peptide).

In additional embodiments, the invention is directed to mixtures of B cells comprising B cells reactive to a specific antigen and B cells not reactive to a specific antigen. In these embodiments, the B cells reactive to a specific antigen are labeled with a tetrameric antigen-avidin complex not comprising an MHC molecule. The antigen on the complex is the specific antigen.

As used herein, a “B cell reactive to a specific antigen” or “specific B cell” is a B cell that comprises surface IgG to a particular antigen of interest, e.g., an antigen of a viral pathogen, or a mammalian antigen targeted in an autoimmune disease.

These cell mixtures can be created, e.g., when B cells from a mammal are evaluated for reactivity to a specific antigen using the complex of the present invention. In preferred embodiments, the mammal is a human patient being tested for the presence of B cells having reactivities relevant to a particular disease or the immune state of the patient. Examples are B cells reacting to a vaccine antigen, a microbial antigen, a viral antigen, a specific antigen targeted in an allergic reaction, or a specific antigen targeted in an autoimmune disease. An example of the latter antigen is a dsDNA mimetope targeted in lupus, such as the R4A peptide having SEQ ID NO:1.

The various compositions described above are useful in various methods for identifying, enumerating, and isolating B cells of specific reactivity.

Thus, in some embodiments, the present invention is directed to methods of labeling a B cell reactive to a specific antigen. The methods comprise combining the B cell with a tetrameric antigen-avidin complex not comprising an MHC molecule, as described above. In these embodiments, the tetrameric antigen-avidin complex comprises the specific antigen and a detectable moiety.

In these methods, the B cell reactive to the specific antigen may be in a mixture of B cells comprising B cells not reactive to the specific antigen. Such mixtures are useful, for example, when analyzing PBMCs from blood for the presence of the specific B cell.

B cells reactive to any specific antigen may be labeled using these methods. The specific antigen may be, for example, a peptide, protein, carbohyhdrate, or hapten antigen. Useful antigens include antigens targeted in an allergic reaction, where B cells reactive to such antigens could predict an allergic response in a subject. The effectiveness of a vaccine can also be determined by identifying B cells, e.g., B memory cells, reactive to the vaccine antigen. Additionally, identification of B cells reactive to a microbial or viral pathogen can be useful in disease diagnosis.

A particularly useful embodiment of these methods employs tetramers comprising a mammalian antigen targeted in an autoimmune disease. This embodiment is useful for, e.g., diagnosing the autoimmune disease, determining the effectiveness of a treatment, or following the progression of the disease. A preferred example of an autoimmune disease that can be evaluated by these methods is lupus, where B cells reactive to the dsDNA mimetope having the sequence of SEQ ID NO:1 are present.

The present invention is also directed to methods of isolating a B cell reactive to a specific antigen from a mixture of B cells reactive to more than one antigen. The methods comprise combining the mixture of B cells with a tetrameric antigen-avidin complex not comprising an MHC molecule, where the tetrameric antigen-avidin complex comprises the specific antigen and a moiety that facilitates isolation of the specific B cell, then isolate the specific B cell. Preferably, the moiety that facilitates isolation of the specific B cell is a fluorescent moiety, where the B cell can then be isolated with a fluorescence-activated cell sorter. However, other moieties could also be used, such as a His6 moiety, allowing isolation of the specific cells with an Ni-nitrilotriacetate matrix. The skilled artisan could select a moiety/isolation method for any particular purpose without undue experimentation.

As with the other methods described above, these methods are not limited to isolation of B cells reactive to any specific antigen. Particularly useful antigens are envisioned to be antigens targeted in an allergic reaction, vaccine antigens, microbial antigens, viral antigen, and, especially, mammalian antigens targeted in an autoimmune disease. As discussed above, a preferred autoimmune disease is lupus, where B cells reactive to the dsDNA mimetope R4A (SEQ ID NO:1) are often present.

These methods may also be useful for isolating B cells reactive to a specific antigen to which hybridomas are subsequently made.

In additional embodiments, the invention is directed to methods of quantifying B cells reactive to a specific antigen in a mixture of B cells reactive to more than one antigen. These methods comprise combining the mixture of B cells with a tetrameric antigen-avidin complex not comprising an MHC molecule, where the tetrameric antigen-avidin complex comprises the specific antigen and a detectable moiety, then counting the B cells comprising the detectable moiety.

As with other methods described above, the detectable moiety in these methods is preferably a fluorescent moiety (e.g., a fluorescent dye), allowing counting of the B cells in a Coulter counter or FACS. However, other detectable moieties can be utilized, for example radioactive moieties, allowing enumeration of the specific B cells by quantifying radioactivity, or antigenic tags, allowing enumeration by microscopic observation after immunological staining.

The invention is additionally directed to methods of measuring immunity in a mammal characterized by the presence of B cells reactive to a specific antigen. The methods comprise combining B cells from the mammal with a tetrameric antigen-avidin complex not comprising an MHC molecule, where the tetrameric antigen-avidin complex comprises the specific antigen and a detectable moiety, then identifying B cells bound to the complex, wherein the B cells bound to the complex are B cells reactive to the specific antigen.

These methods are not limited to any particular mammal. In preferred embodiments, the mammal is a human or an experimental animal.

In most embodiments, the B cells for these methods are preferably obtained from the blood of the mammal. However, B cells could also be obtained from a tissue (e.g., spleen) biopsy.

In some embodiments of these methods, the B cells bound to the complex are counted, since more B cells reactive to the specific antigen generally indicates a greater degree of immunity to the antigen. However, in other embodiments, such as in determining whether an individual has an autoimmune disease or an allergy, the presence of any B cells reactive to the specific antigen is diagnostic, and counting the specific B cells is not necessary. In the latter case, the B cells bound to the complex can be visualized microscopically.

These methods are particularly useful for evaluating the effectiveness of a vaccine where the mammal has been immunized with the specific antigen. The methods are also useful for determining whether the mammal is potentially allergic to the specific antigen. Additionally, these methods can be used to determine if the mammal has an immunity to a pathogen (e.g., a virus or a microbe), to ascertain if the mammal has been exposed or is infected with the pathogen, or whether a vaccine against the pathogen that has been administered to the mammal is effective.

These methods can also be used to determine whether the mammal has B cells reactive to a specific antigen targeted in an autoimmune disease such as lupus. As discussed above, a particularly useful antigen for evaluating lupus is the dsDNA mimetope comprising the sequence of SEQ ID NO:1 (the R4A peptide).

The present invention is additionally directed to methods of monitoring a disease in a mammal or determining whether a mammal has a disease characterized by the presence of B cells reactive to a specific antigen. The methods comprise combining B cells from the mammal with a tetrameric antigen-avidin complex not comprising an MHC molecule, where the tetrameric antigen-avidin complex comprises the specific antigen and a detectable moiety, then identifying B cells bound to the complex, where the presence of B cells bound to the complex indicates the presence of the disease in the mammal.

In preferred embodiments of these methods, the mammal is a human or an experimental animal.

Examples of diseases that can be diagnosed or monitored using these methods are allergies and autoimmune diseases such as lupus. As discussed above, lupus can be diagnosed or monitored by identifying B cells reactive to the peptide dsDNA mimetope having SEQ ID NO:1.

As with the other methods described above, preferred detectable moieties are fluorescent dyes, but other detectable moieties may be useful for various applications.

The B cells bound to the complex can also be counted in these embodiments, since severity of disease would likely be correlated with the number of specific B cells. Thus, for example, increasing numbers of B cells reactive to the peptide dsDNA mimetope (as measured by, e.g., counting B cells that comprise bound peptide-avidin tetramer) would be expected to be associated with more severe lupus. Treatments for the disease can thus be evaluated by quantifying the specific B cells at various times during treatment, where an effective treatment would be expected to cause a decrease or elimination of the specific B cells in the patient.

Preferred embodiments of the invention are described in the following Example. Other embodiments within the scope of the claims herein will be apparent to one skilled in the art from consideration of the specification or practice of the invention as disclosed herein. It is intended that the specification, together with the Example, be considered exemplary only, with the scope and spirit of the invention being indicated by the claims which follow the Example.

### EXAMPLE

Identification of an Antigen-Specific B Cell Population

### Example Summary

The difficulty in characterizing antigen-specific B cells that arise in the native B cell repertoire has been a formidable obstacle to understanding both protective and pathogenic antibody responses. We have developed a tetramer-based technique for identifying antigen-specific B cells. Biotin-labeled antigen is made tetrameric by interaction with streptavidin. The enhanced avidity of this antigenic compound for the B cell membrane permits the visualization, characterization and isolation of antigen-specific B cells.

### Introduction

Studies of antigen-specific T cells have been facilitated by the use of tetrameric peptide-MHC molecules that will bind to T cell receptors specific for that peptide-MHC complex. The avidity enhancement that is mediated by the tetrameric molecule has proven to be critical in permitting high sensitivity and specificity in the identification of antigen-specific T cell populations. We have adapted this avidity-enhanced methodology to the detection of antigen-specific B cells. We have identified anti-DNA B cells in two murine models and anti-PC B cell in two other murine models. These studies support the feasibility of this approach, and suggest it can be adapted to any B cell response.

We have previously reported a peptide sequence (R4A peptide) that behaves as a dsDNA mimetope (Gaynor et al., 1997). Immunization of BALB/c mice with a multimeric form of this peptide can induce elevated serum titers of anti-DNA antibodies (Putterman and Diamond, 1998). To identify B cells participating in this response, we developed a tetrameric fluorochrome-labeled form of the R4A peptide (R4A-tetramer) which has greater avidity than monomer for peptide-reactive B cells. Using this reagent, we can identify autoreactive, antigen-specific B cells in two mouse models with anti-DNA antibody production. The first model involves immunization of non-autoimmune BALB/c mice with a multimeric form of this peptide (MAP-peptide), resulting in the T cell-dependent production of IgG anti-dsDNA antibodies which deposit in renal glomeruli (Putterman and Khalil). R4A-tetramers were used to identify the pathogenic B cells in MAP-peptide immunized mice.

We also demonstrate the utility of this tetramer to identify antigen-specific B cells in mice transgenic for the γ2b heavy chain of the R4A antibody. In this model, approximately 5% of B cells express the transgene. We have previously demonstrated the expansion and activation of anti-DNA B cells in R4Aγ2b transgenic BALB/c mice that are exposed to serum levels of estradiol of 75-100 pg/ml for a 4-6-week duration. Autoreactive B cells from estradiol-treated mice can be identified with tetrameric peptide.

Finally, the laboratory has reported two peptide mimetopes of PC. In each case, these mimetopes can induce an anti-PC response in immunized mice, although the elicited antibodies do not protect against a lethal pneumococcal infection (Harris et al. 2000, 2002). Tetrameric configurations of these peptides recognize antigen-specific B cells.

### Materials and Methods

Tetramer Generation.

Peptide-neutravidin-R-phycoerythrin tetramers and peptide-streptavidin-allophycocyanin tetramers were generated by combining an equal volume of N-terminal biotinyl peptide at 200 μM (Research Genetics, Huntsville, Ala.) with R-phycoerythrin-labeled neutravidin at 3.3 μM (PE-NA, Molecular Probes, Eugene, Oreg.), or allophycocyanin-labeled streptavidin at 6.1 μM (APC-SA, Molecular Probes). Each mixture was incubated at 4° C. for a minimum of 3 h. Subsequently, peptide-PE and peptide-APC complexes were separated from uncomplexed peptide by gel filtration using a Bio-Gel P-30 spin column (Bio Rad, Hercules, Calif.). Complexes with unlabeled neutravidin (Southern Biotechnology, Birmingham, Ala.) and streptavidin (Pierce, Rockford, Ill.) were similarly generated and used as inhibitors. Protein concentration of the purified tetramer solutions was determined using the BCA protein assay kit (Pierce) and a bovine serum albumin standard. The tetramer solutions were centrifuged at 20,000×g for 10 min prior to use to remove protein aggregates. Peptides used were R4A peptide (DWEYSLWLSN) (Gaynor et al., 1997)(SEQ ID NO:1), 10-2 peptide (ADGSGGRDEMQASMWS) (Harris et al., 2002)(SEQ ID NO:2) and peptide 1 (ASRNKANDYTTEYSASVKGRFIV) (Kang et al., 1988; Harris et al. 2000)(SEQ ID NO:3).

Generation of Fluorescent PC.

Phosphorylcholine (PC) coupled to BSA (5 PC per BSA, Biosearch Technologies, Novato, Calif.) was fluorescently labeled using an Alexa Fluor 647 protein-labeling kit according to the manufacturer's protocol (Molecular Probes).

Mice.

Six- to eight-week-old female BALB/c mice (Jackson Laboratory, Bar Harbor, Me.) were used in all studies except those involving transgenic mice and were housed in accord with AALAC regulations. Mice were immunized intraperitoneally on day 0 with a 100 μl of a 1:1 emulsion of Complete Freund's Adjuvant (CFA, Difco Laboratories, Detroit, Mich) containing 100 μg of R4A peptide on a branched polylysine backbone (Research Genetics), or 10-2 and peptide 1 peptide coupled to BSA. Sham-immunized mice were immunized with 100 μg of the MAP-backbone alone (MAP™; Applied Biosystems, Foster City, Calif.) or BSA alone in CFA. On days 7 and 14, experimental mice were boosted intraperitoneally with 100 μg MAP-peptide or peptide BSA in incomplete Freund's adjuvant (IFA), while control mice were boosted with either MAP-backbone or BSA emulsified in IFA, or IFA alone. Spleens were harvested for analysis 5-7 days after the last immunization. Analysis of the primary anti-peptide response was performed on mice 16 days after immunization with a single 100 μg dose (100 μl total volume) of MAP-peptide emulsified in CFA.

R4A-γ2b transgenic BALB/c mice were bred in the institutional animal facility. 17β-estradiol (Innovative Research of America, Sarasota, Fla.) or placebo pellets were surgically implanted in 6- to 8-week-old female mice. Estradiol pellets contained 0.18 mg 17β-estradiol designed for release over 60 days. Spleen cells were harvested 4-6 weeks later.

Flow Cytometry.

Spleens were harvested and dissociated into single-cell suspensions by passage through a 70 μm nylon mesh. Erythrocytes were lysed in 0.17 M NH₄OH, pH 7.3. The resultant cell suspensions were stained in phosphate-buffered saline (PBS), pH 7.3 with premixed combinations of fluorochrome-labeled antibodies, tetramers, or PC-BSA at optimal dilutions. Staining was routinely conducted at 4° C. for 1 h. Inhibitor was added to cell suspensions prior to addition of the staining cocktail at a 10-fold molar excess above the concentration of labeled tetramer. Stained cells were washed and resuspended in PBS containing 1 μg/ml of propidium iodide. Cells were also stained with APC-anti-mouse CD45R (B220, clone RA3-6B2, Caltag Laboratories, Burlingame, Calif.), and PE-anti-mouse CD 19 (clone 1D3, Becton Dickinson, Pharmingen).

Data were acquired using a FACSCalibur flow cytometer and Cell Quest software (BD Immunocytometry Systems). Between 200,000 and 250,000 events were acquired per sample. All data are representative plots derived from a minimum of three independent experiments in which at least three experimental and three control mice were analyzed. Analysis was performed using FlowJo software (Tree Star, San Carlos, Calif.). Bivariate plots are presented as 5% probability contours with outliers, and displayed on a log scale.

Purification of Tetramer-Binding B Cells.

Spleens were harvested from BALB/c mice immunized intraperitoneally with R4A peptide as described. The final immunization took place 7 days prior to sacrifice. Single-cell suspensions were prepared as described above in sterile Hanks Balanced Salt Solution without divalent cautions (HBSS, Life Technologies, Rockville, Md.). Erythrocytes were lysed in 0.17 M NH₄OH, pH 7.3. The cells were washed, filtered through a sterile 40 μm mesh and resuspended at a concentration of 0.5-1.0×10⁸ cells/ml in HBSS. R4A-PE was added to the suspensions at a final concentration of 3 nM and incubated for 30 min at 4° C. The cells were washed and resuspended in HBSS supplemented with 0.5% bovine serum albumin (Fraction V, Roche, Indianapolis, Ind.) and 2 mM EDTA (Sigma, St. Louis, Mo.) at a concentration of 1.25×10⁸ cells/ml. Anti-PE microbeads (Miltenyi, Auburn, Calif.) were added (200 μl beads/10⁸ cells) to the suspension and incubated for 15 min at 4° C. The cells were washed, resuspended at a final concentration of 2×10⁸ ml and run through a VS+positive enrichment column mounted on a VarioMACS magnet (Miltenyi) according to the manufacturer's protocol. The cell fraction enriched for R4A-tetramer reactivity was then either put directly into culture or stained with appropriate reagents for flow cytometry and sorted into subfractions on a FACS-Star Plus cell sorter or a FACS Vantage SE cell sorter (BD Immunocytometry Systems).

The sorted, peptide-reactive populations were obtained at greater than 80% purity, with 3% or lower contamination from the peptide-nonreactive B220⁺ gate. Both peptide-nonreactive populations were obtained at greater than 95% purity.

RT-PCR.

RNA was isolated using TRIzol (Invitrogen, Carlsbad, Calif.) according to the manufacturer's instructions. The subsequent RNA pellet was briefly dried and resuspended in DEPC-treated water. cDNA was prepared using the Superscript II 1st Strand Synthesis Kit (Invitrogen) according to the manufacturer's protocol. A 2 μl RNA sample (representing approximately 40,000 sorted cells) was combined with 150 ng random hexamers, 1 mM dNTPs, and 4 μl DEPC-treated water and heated to 68° C. for 5 min, then chilled on wet ice. To each sample, 2 μl of 10× First Strand Buffer, 5 mM MgCl₂, 10 mM dithiothreitol and 40 U RNAase Out were added, and the solution was incubated 2 min at 25° C. Following incubation, 50 U of Superscript II was added and the sample was incubated for 10 min at 25° C., followed by 50 min at 42° C. The reaction was stopped by incubation at 68° C. for 15 min. Finally, 2 U RNAse H was added and the samples incubated for 20 min at 37° C. to remove RNA/DNA heterodimers.

PCR.

PCR for CD79b (Igβ) was performed on 2 μl cDNA template prepared above, using HotStarTaq Mastermix (Qiagen, Valencia, Calif.) according to the manufacturer's protocol. Primers were used at a final concentration of 400 nM. The primers used for CD79b are based on published sequences (Lin and Grosschedl, 1995) as follows: B29_pA 5′-GGTGAGCCGGTACCAGCAATG-3′ (SEQ ID NO:4), B29_pB 5′-AGTTCCGTGCCACAGCTGTCG-3′ (SEQ ID NO:5). These primers span an intron to avoid possible amplification from DNA.

Multiplex PCR was performed using the QuantumRNA Classic 18S Internal Standard (Ambion, Austin, Tex.) as a control to determine relative expression levels. This kit provides primers for 18S ribosomal RNA used as an internal control because of its invariant expression across cell types. The kit also includes “comptimers” (18S-specific primers that cannot be extended) in order to reduce 18S amplification to a level comparable to that of the gene of interest. CD79b primer was used at a final concentration of 0.4 μM and combined with 18S rRNA primers and comptimers in a 2:8 ratio to obtain similar quantities of the amplified PCR products. PCR was performed in 50 μl volume in a GeneAmp PCR System 9700 (Applied Biosystems). Following 15 min at 94° C. to activate the HotStarTaq polymerase, 37 cycles were performed as follows: 94° C. 30 s, 55° C. 30 s, 72° C. 60 s. The number of cycles was chosen to avoid saturation. The reaction was followed by a 7 min extension step at 72° C., then cooled to 4° C. Products were analyzed on a 2% agarose gel, and visualized using ethidium bromide.

The PCR products were quantitated from digital images generated by scanning the film with a computing densitometer (Molecular Dynamics, Sunnyvale, Calif.). Images were analyzed using Scion Image (Scion). The relative expression level of CD79b in each sorted cell population was determined by comparing the ratio of CD79b intensity to 18S intensity within individual PCR reactions.

ELISpot Assay.

Isolated tetramer-binding B cells and non-binding B cells were cultured in vitro for 48 h in RPMI 1640 medium (Life Technologies) with 10% fetal calf serum (Hyclone Laboratories, Logan, Utah) 100 U/ml penicillin and 100 μg/ml streptomycin (Sigma), and 50 μM 2-mercaptoethanol (Sigma) supplemented with 20 μg/ml lipopolysaccharide from Escherichia coli serotype 055:B5 (LPS, Sigma). After 48 h of culture in the presence of LPS, cells were counted in the presence of trypan blue (Life Technologies) and were plated onto antigen-coated microtiter plates in culture medium. The plates were spun briefly and then cultured for 6 h at 37° C. in a humidified incubator with 10% CO₂. Biotinylated polyclonal-goat-anti-mouse IgG (Southern Biotechnology) diluted to 1 μm/ml in BSA/PBS was added to the plates and incubated for 1 h at 25° C. The plates were washed and incubated with a solution of 1 μg/ml streptavidin-alkaline phosphatase (Southern Biotechnology) for 1 h at 25° C. The plates were developed with a 1 mg/ml solution of 5-bromo-4-chloro-3-indolyl phosphate, p-toluidine salt (Sigma) in AMP buffer [9.58% 2-amino-2-methyl-1-propanol, 1 mM MgCl₂, 0.01% Triton-X-405 (Sigma), pH 9.8]. The plates were allowed to develop until spots became apparent and the reaction was stopped by washing the plates with H₂O. Spots were counted manually using a dissecting microscope. Assays were performed in triplicate and repeated three times.

ELISA.

Microtiter plates were coated with either peptide 1, 10-2, or goat anti-mouse IgG overnight, and blocked with 5% milk powder. Supernatants from in vitro cultures were collected and added to the plates and incubated 1 h at 37° C. The plates were washed and incubated with a solution of 1 μg/ml goat anti-mouse IgG coupled to alkaline phosphatase for 1 h at 37° C. Plates were developed using 1 mg/ml p-nitrophenyl phosphate (Sigma 104 phosphatase substrate tablets; Sigma) in 1 mM MgCl₂ and 50 mM NaHCO₃ as needed at room temperature. Optical density was read at 405 nm. Antibody quantities were normalized to total IgG.

Statistics.

Statistical comparisons of means were made using the unpaired Student's t-test function in Prism (Graph Pad Software).

### Results

Splenocytes from BALB/c Mice Immunized with MAP-Peptide Specifically Stain with R4A-Tetramer.

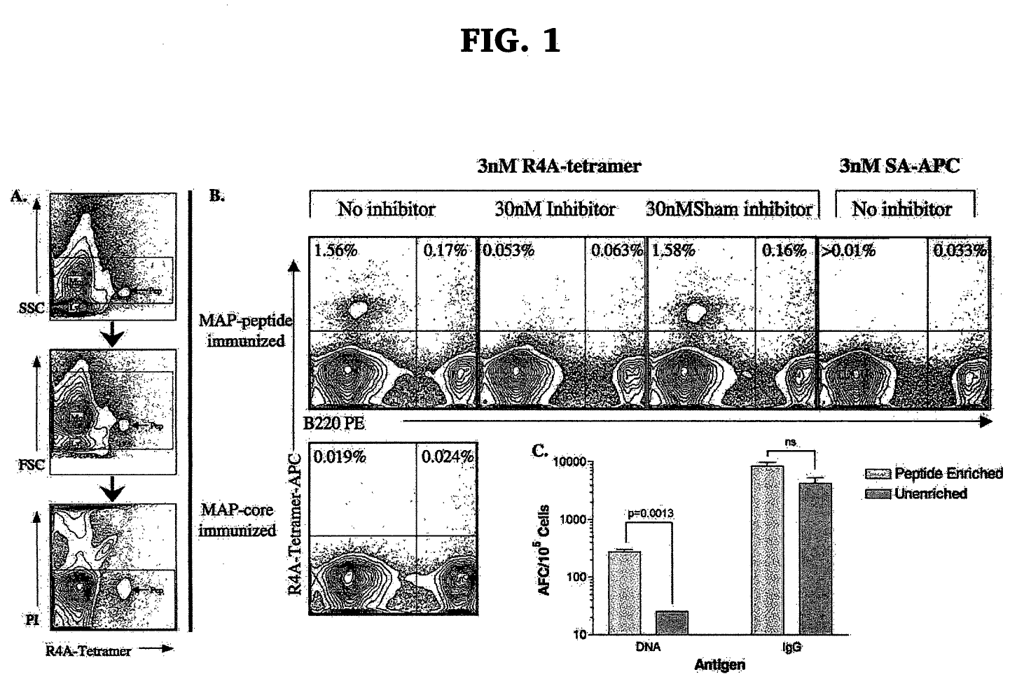

To activate an antigen-specific B lymphocyte population, BALB/c mice were immunized following a protocol previously demonstrated to elicit an anti-peptide, anti-dsDNA cross-reactive response (Putterman and Diamond, 1998). R4A-tetramer was used to identify peptide-reactive cells. Since we expected only a small population of tetramer-reactive lymphocytes, scatter gates were set to exclude extraneous populations (FIG. 1A). Propidium iodide-permeable (dead) cells were also excluded from the analyses.

MAP-peptide immunization with R4A peptide led to high titers of serum reactivity to peptide and dsDNA and to a frequency of cells staining with peptide that was approximately 1% of gated cells (FIG. 1 B). Mice immunized with MAP-core or with adjuvant alone had no serum anti-peptide or anti-DNA reactivity and demonstrated very low numbers of R4A-tetramer-binding cells that did not exceed 0.05% of the gated population, similar to background reactivity with streptavidin-allophycocyanin (FIG. 1B) and consistent with a low precursor frequency of antigen-specific B lymphocytes in the pre-immune host.

The specificity of tetramer binding was demonstrated by inhibition studies with unlabeled R4A-tetramer complexes. Splenocytes were preincubated with either unlabeled tetramers or unlabelled streptavidin (sham inhibitor) followed by incubation with labeled tetramers and anti-B220 antibody. A 10-fold excess of inhibitor was used. As shown in FIG. 1B, tetramer staining was nearly abolished by inclusion of unlabeled tetramers. Monomeric peptide did not inhibit the binding of labeled tetramer. The peptide-binding cells did not express T cells, monocyte or dendritic cells markers (data not shown).

To demonstrate that tetramer-reactive B cells could produce anti-DNA antibody, we isolated tetramer-reactive cells and assayed for anti-DNA antibody production after LPS stimulation. The tetramer-enriched fraction and the non-enriched population possessed equal percentages of IgG-producing cells. However, the tetramer-enriched population demonstrated a 10-fold higher frequency of anti-DNA B cells (FIG. 1C).

We have previously reported that R4A-2b transgenic mice treated with estradiol develop serum titers of anti-dsDNA antibody (Bynoe et al., 2000). Estradiol treatment of these mice results in an increase in the total number of splenocytes; however, the percentage of B220⁺ cells in the spleen is unaltered. B lymphocytes expressing the R4A transgene, identified by their expression of γ2b, are significantly expanded in estradiol-treated mice (Bynoe et al., 2000). Previous experiments have demonstrated that essentially all γ2b-expressing B lymphocytes express the R4A transgene (Iliev et al., 1994; Spatz et al. 1997).

We asked whether the R4A-tetramer could identify antigen-specific B cells in this autoimmune model which is mediated by a small number of autoreactive lymphocytes. Mice were treated with estradiol for 5 weeks. R4A-tetramer staining in estradiol-treated mice revealed an expansion in both γ2b and tetramer-reactive B cells. R4A-tetramer-reactive B cells expanded twofold over placebo control (p=0.0207). This is consistent with our previous demonstration of an increase in DNA-reactive B cells, measured in an antigen-specific ELISpot assay and by hybridoma formation (Bynoe et al., 2000; Peeva et al., 2000).

Together, these two models demonstrate the ability of tetramer technology to identify antigen-specific B cells in a response mediated by a physiological or small number of cells, and without the potential distortions inherent in the generation of hybridomas or the altered maturation and migration possible in transgenic models which involve large numbers of autoreactive B cells.

Splenocytes from BALB/c Mice Immunized with Peptide Mimetopes of PC Specifically Stain with Peptide-Tetramers.

Our laboratory recently reported two peptide mimetopes of phosphorylcholine (PC), the dominant epitope of Streptococcus pneumoniae cell wall polysaccharide (Harris et al., 2000, 2002). Although immunization with these two peptides, designated 10-2 and peptide 1, does not induce a protective response, both result in anti-peptide and anti-PC antibody responses. In each case, the anti-peptide response is specific and not cross-reactive with the other mimetope.

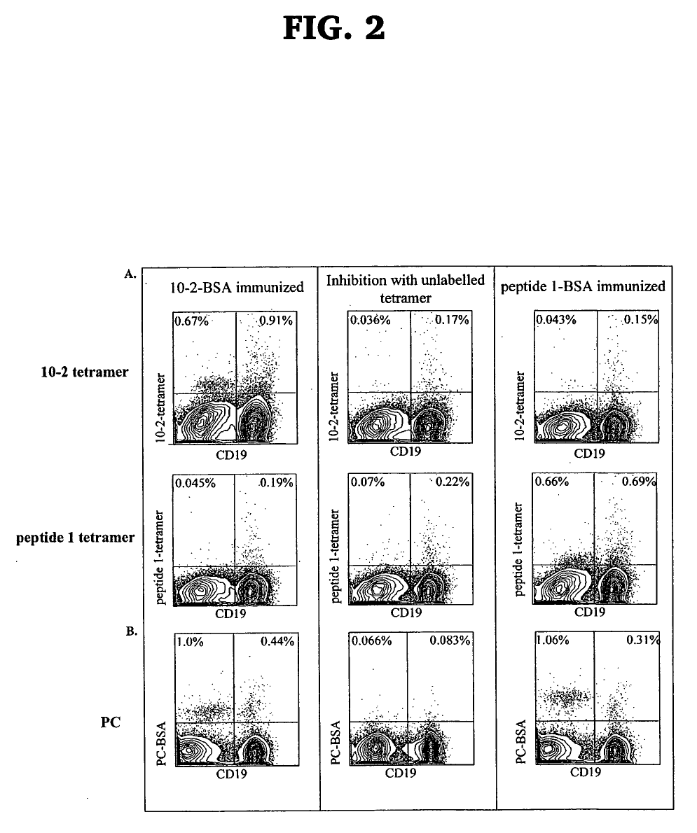

In order to determine whether peptide-tetramers could routinely recognize an antigen-specific B lymphocyte population arising in a non-autoreactive immune response, we immunized mice with each peptide coupled to BSA. Fluorochrome-labeled tetramers of each peptide were made and used to identify antigen-specific cells. We were able to identify peptide-specific B lymphocytes in mice immunized with each peptide (FIG. 2A). Additionally, 10-2 tetramers did not label B cells from peptide 1-BSA-immunized mice, and vice versa. This corresponds with the lack of serum cross-reactivity observed (data not shown).

To demonstrate the specificity of staining, inhibition was performed with unlabeled tetramer complexes. As shown in FIG. 2A, the presence of specific unlabeled tetramer reduced fluorochrome-tetramer binding to background level.

Splenocytes from mice immunized with each PC mimetope were assayed for their ability to bind a non-tetrameric, fluorescent conjugate of PC-BSA. This conjugate has five PC molecules per BSA molecule and thus is a multimeric compound. We were able to identify PC-specific splenocytes in mice immunized with each peptide. Unlabeled PC-BSA completely ablated staining, demonstrating the specificity of this method (FIG. 2B).

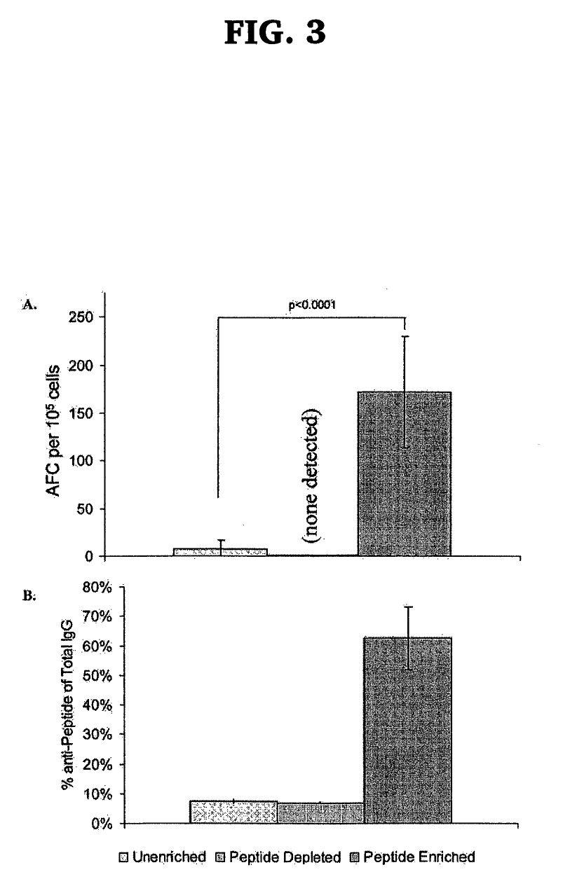

To demonstrate again the ability of antigen-specific splenocytes to produce antigen-specific antibody, we sorted peptide 1 tetramer-reactive splenocytes from immunized mice and assayed for antigen-specific antibody-secreting cells by ELISpot assay. The tetramer-reactive population showed significant enrichment for cells making antibodies to peptide 1 compared to unsorted or tetramer-negative populations (FIG. 3A). Additionally, supernatants from cultures of tetramer-reactive splenocytes showed significant enrichment for anti-peptide antibody (FIG. 3B).

Peptide-Reactive Splenocytes Retain Expression of CD79b, a B Cell Specific Gene.

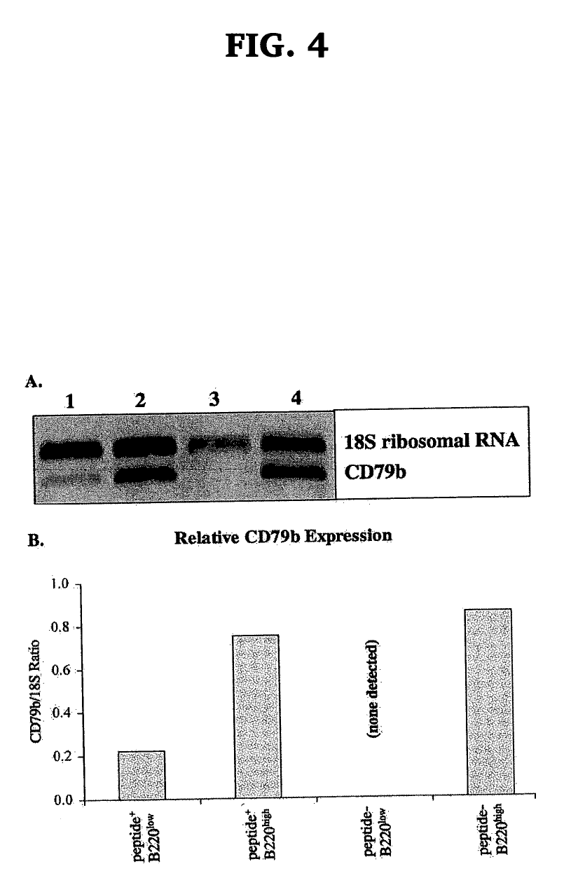

Although the tetramer-reactive population does not express typical T cell, macrophage, or monocyte markers, some typical B cells markers such as B220 (FIG. 1B), CD19 and CD79b (McHeyzer-Williams et al., 2000 and data not shown) are expressed at reduced levels in a subpopulation of the tetramer-reactive cells. While there is a B220^{high} population that expresses the lineage-specific markers CD19 and CD79b, there is a also B220^{low} population that binds tetramers. To show that this population includes B cells, we determined surface expression of CD19 and CD79b. Because expression of these markers was low, we were concerned that this tetramer-reactive population might be due to passive absorption of tetramer-specific antibodies by Fc receptors on macrophages. To address this issue, tetramer-reactive splenocytes were isolated as described above. RNA was isolated from the sorted splenocytes and RT-PCR for a B cell-specific gene, CD79b, was performed (FIG. 4A). CD79b expression was normalized to an internal standard within each population. Consistent with our FACS data and other published studies (McHeyzer-Williams et al., 2000), tetramer-specific B220^{low} (FIG. 1B, upper left quadrant) splenocytes retain CD79b mRNA expression, albeit at a reduced level compared to B220^{high} splenocytes (FIG. 4B). Expression of CD79b in the tetramer-specific B220^{high} population was similar to that in the tetramer-negative B220^{high} population, whereas little or no CD79b expression was observed in the B220^{low} cells that did not bind tetramers. This clearly demonstrates that the peptide-specific population contains a significant number of B cells.

Discussion

We have demonstrated the ability of peptide-tetramers to identify antigen-specific B cells. This technique offers several advantages over prior methods. Unlike hybridoma production, the technique is rapid and able to represent the entire population of B cells, not only those able to form stable hybridomas. Antigen-specific responses can be studied using several transgenic models, but there is the concern that transgenic systems may alter the maturation, migration and selection of B cells in an antigen-specific response. Furthermore, the generation of transgenic models is time consuming and labor intensive. We demonstrate that fluorescent, tetrameric antigen is easily synthesized and may be used to follow individual antigen-specific cells without perturbing the kinetics of the immune response. This methodology should help reveal important features of humoral immune responses to different antigens in different hosts.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

Appendix—SEQ ID Nos.

SEQ ID NO:1-R4A peptide
DWEYSLWLSN
SEQ ID NO:2-10-2 peptide
ADGSGGRDEMQASMWS
SEQ ID NO:3-peptide 1
ASRNKANDYTTEYSASVKGRFIV
SEQ ID NO:4-CD79b primer B29_pA
GGTGAGCCGGTACCAGCAATG
SEQ ID NO:5-CD79b primer B29_pB
AGTTCCGTGCCACAGCTGTCG

1. A tetrameric antigen-avidin complex not comprising an MHC molecule.

2. (canceled)

3. The complex of claim 1, labeled with detectable moiety.

4. The complex of claim 3, wherein the detectable moiety is a fluorescent molecule.

5-6. (canceled)

7. The complex of claim 1, wherein the antigen is a mammalian antigen targeted in an autoimmune disease.

8. The complex of claim 7, wherein the autoimmune disease is lupus.

9. (canceled)

10. The complex of claim 1, wherein the antigen is a peptide that is a dsDNA mimetope.

11. The complex of claim 10, wherein the peptide comprises the sequence of SEQ ID NO:1.

12. (canceled)

13. A mammalian B cell comprising the tetrameric antigen-avidin complex of claim 1.

14. The B cell of claim 13, wherein the B cell is a human B cell.

15. The B cell of claim 13, wherein the complex further comprises a fluorescent moiety.

16. The B cell of claim 13, wherein the antigen is a dsDNA mimetope.

17. The B cell of claim 15, in a composition further comprising B cells that do not comprise the complex.

18. The B cell of claim 15, isolated from B cells not comprising the complex.

19-20. (canceled)

21. A reagent for diagnosing an autoimmune disease or monitoring progression of an autoimmune disease, the reagent comprising the tetrameric antigen-avidin complex of claim 1, wherein the antigen is a dsDNA mimetope.

22. The reagent of claim 21, wherein the autoimmune disease is lupus and the antigen comprises SEQ ID NO:1.

23-28. (canceled)

29. A method of labeling a B cell reactive to a specific antigen, the method comprising combining the B cell with the tetrameric antigen-avidin complex of claim 1, wherein the tetrameric antigen-avidin complex comprises the specific antigen and a detectable moiety.

30-35. (canceled)

36. A method of isolating a B cell reactive to a specific antigen from a mixture of B cells reactive to more than one antigen, the method comprising
combining the mixture of B cells with the tetrameric antigen-avidin complex of claim 1, wherein the tetrameric antigen-avidin complex comprises the specific antigen and a fluorescent moiety,
then isolating the B cell with a fluorescence-activated cell sorter.

37-41. (canceled)

42. A method of quantifying B cells reactive to a specific antigen in a mixture of B cells reactive to more than one antigen, the method comprising
combining the mixture of B cells with the tetrameric antigen-avidin complex of claim 1, wherein the tetrameric antigen-avidin complex comprises the specific antigen and a detectable moiety,
then counting the B cells comprising the detectable moiety.

43. (canceled)

44. A method of measuring immunity in a mammal characterized by the presence of B cells reactive to a specific antigen, the method comprising
combining B cells from the mammal with the tetrameric antigen-avidin complex of claim 1, wherein the tetrameric antigen-avidin complex comprises the specific antigen and a detectable moiety, then
identifying B cells bound to the complex,
wherein the B cells bound to the complex are B cells reactive to the specific antigen.

45-57. (canceled)

58. A method of monitoring a disease in a mammal or determining whether a mammal has a disease, wherein the disease is characterized by the presence of B cells reactive to a specific antigen, the method comprising
combining B cells from the mammal with the tetrameric antigen-avidin complex of claim 1, wherein the tetrameric antigen-avidin complex comprises the specific antigen and a detectable moiety, then
identifying B cells bound to the complex,
wherein the presence of B cells bound to the complex indicates the presence of the disease in the mammal.

59-66. (canceled)


SEQUENCE LISTING (5 sequences)

SEQ ID NO: 1 - protein, 10 aa, Artificial
  synthesized R4A peptide
DWEYSLWLSN

SEQ ID NO: 2 - protein, 16 aa, Artificial
  synthesized 10-2 peptide
ADGSGGRDEMQASMWS

SEQ ID NO: 3 - protein, 23 aa, Artificial
  synthesized peptide 1
ASRNKANDYTTEYSASVKGRFIV

SEQ ID NO: 4 - DNA, 21 bp, Artificial
  CD79b primer B29_pA
ggtgagccggtaccagcaatg

SEQ ID NO: 5 - DNA, 21 bp, Artificial
  CD79b primer B29_pB
agttccgtgccacagctgtcg